(19) United States
(12) Patent Application Publication
(10) Pub. No.: US 2013/0217986 A1
(43) Pub. Date: Aug. 22, 2013

(54) METHOD AND SYSTEM FOR PREDICTING AN AMYLOIDOSIS STATUS

(71) Applicant: UCL Business plc, (US)

(72) Inventors: Imre Lengyel, London (GB); Tunde Peto, London (GB)

(73) Assignee: UCL BUSINESS PLC, London (GB)

(21) Appl. No.: 13/772,600

(22) Filed: Feb. 21, 2013

Related U.S. Application Data
(60) Provisional application No. 61/601,362, filed on Feb. 21, 2012.

(51) Int. Cl. A61B 5/00; A61B 3/12; A61B 5/1455

(52) U.S. Cl. CPC A61B 5/4088; A61B 5/1455; A61B 3/12; A61B 5/7282
USPC 600/318

(57) ABSTRACT

An exemplary method of predicting an amyloidosis status involves obtaining a profile of drusen in a periphery of a retina of a subject; and using the profile to predict an amyloidosis status. Another exemplary method of predicting an amyloidosis status involves identifying a subject, the amyloidosis status of which is desired; and obtaining a profile of drusen in a periphery of a retina of the subject. Another exemplary method of predicting an amyloidosis status involves analyzing a profile of drusen in a periphery of a retina of a subject to predict an amyloidosis status of the subject. Another exemplary method of predicting an amyloidosis status involves comparing a profile of drusen in a periphery of a retina of a subject to a control; and predicting an amyloidosis status of the subject.

### RELATED APPLICATIONS

This application claims priority from U.S. Provisional Application Ser. No. 61/601,362 filed Feb. 21, 2012, the entire disclosure of which is incorporated herein by this reference.

### TECHNICAL FIELD

The presently-disclosed subject matter relates the diagnosis, prognosis, and monitoring of amyloidosis, especially disorders in which extracellular deposits are formed within the CNS. Such disorders include, for example, Alzheimer's disease, Huntington's disease, and Parkinson's disease. In particular, the presently-disclosed subject matter relates to methods and systems for predicting an Alzheimer's disease status in a subject.

### INTRODUCTION

Alzheimer's disease (AD) is the most common neurodegenerative disorder, affecting about half of all people over the age of 80 years. The prevalence of AD rises steeply with age, and current projections suggest that the number of elderly will double in the next generation. Costs of AD have been estimated to exceed that of heart disease, stroke, and cancer combined. AD is characterized by the misfolding of the beta Amyloid protein to form neurotoxic oligomers before being detoxified in the characteristic amyloid plaque. Several biomarkers of early disease are available with the current gold-standard being positron emission tomography (PET) studies of amyloid deposition and brain metabolism, magnetic resonance imaging (MRI) of cerebral atrophy and CSF tau sampling. (11). However such techniques are invasive or require significant patient co-operation and compliance. In the case of MRI, elderly patients are often unable to have a scan because of contraindications. Furthermore, it is not feasible to undertake serial PET and CSF studies due to restrictions on radiopharmaceutical dosing and the undesirability of repeated CSF sampling. Therefore there is an unmet medical need for technology useful to make Alzheimer's disease assessments in a subject readily, regularly, and with limited invasiveness. Similar needs arise with many if not all of the other diseases falling within the category of amyloidosis.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes methods and systems for predicting an amyloidosis status. In some embodiments, a method is provided, including obtaining a profile of drusen in a periphery of a retina of a subject; and using the profile to predict an amyloidosis status.

In some embodiments, a method is provided, including identifying a subject, the amyloidosis status of which is desired; and obtaining a profile of drusen in a periphery of a retina of the subject. In some embodiments, the method also includes using the profile to predict an amyloidosis status.

In some embodiments, a method is provided, including analyzing a profile of drusen in a periphery of a retina of a subject to predict an amyloidosis status of the subject. In some embodiments, a method is provided, including comparing a profile of drusen in a periphery of a retina of a subject to a control; and predicting an amyloidosis status of the subject. In some embodiments, the method also includes using a system for predicting an amyloidosis status, comprising: data storage means; amyloidosis status program modules stored on the data storage means; a processor means executing the amyloidosis status program modules: obtaining a profile of drusen in a periphery of a retina of a subject; and predicting an amyloidosis status based on the profile.

In some embodiments, a system is provided, including a machine for obtaining a profile of drusen in a periphery of a retina of a subject; and a control of amyloidosis status.

In some embodiments, a system is provided, including data storage means; amyloidosis status program modules stored on the data storage means; a processor means executing the amyloidosis status program modules: obtaining a profile of drusen in a periphery of a retina of a subject; and predicting an amyloidosis status based on the profile. In some embodiments, the system also includes a machine for obtaining a profile of drusen in a periphery of a retina of a subject. In some embodiments of the system, the amyloidosis status program modules comprise a control.

In some embodiments of the presently-disclosed subject matter, predicting an amyloidosis status comprises making a diagnosis. In some embodiments, predicting an amyloidosis status comprises making prognosis. In some embodiments, predicting an amyloidosis status comprises making an assessment of risk.

In some embodiments of the presently-disclosed subject matter, the profile comprises a quantitative assessment of drusen in the periphery of the retina of the subject. In some embodiments, the profile comprises a qualitative assessment of drusen in the periphery of the retina of the subject. In some embodiments, the profile comprises a presence or absence of drusen in the periphery of the retina of the subject. In some embodiments, the profile comprises an amount of drusen in the periphery of the retina. In some embodiments, the profile comprises a size of drusen in the periphery of the retina. In some embodiments, the profile comprises a pattern of drusen in the periphery of the retina. In some embodiments, the profile comprises a location of drusen in the periphery of the retina. In some embodiments, the profile comprises a distribution of drusen in the periphery of the retina.

In some embodiments of the presently-disclosed subject matter, the method and/or system includes comparing the profile to a control. In some embodiments, the control comprises a profile of drusen associated with a known amyloidosis status. In some embodiments, the known amyloidosis status is a diagnosis of amyloidosis. In some embodiments, the known amyloidosis status is a diagnosis of no amyloidosis. In some embodiments, the known amyloidosis status is a known stage of amyloidosis.

In some embodiments of the presently-disclosed subject matter, the method and/or system includes obtaining a subsequent profile of drusen in the periphery of a retina of a subject. In some embodiments, the method and/or system includes predicting an amyloidosis status based on the drusen profiles and/or change in the drusen profiles. In some embodiments, the method and/or system includes comparing the profile and the subsequent profile. In some embodiments, the method and/or system includes identifying changes, if any, between obtained profiles of drusen. In some embodiments, the method and/or system includes predicting an amyloidosis status based on the identified changes, or lack thereof In some embodiments, the amyloidosis status is no substantial change, a progression of amyloidosis, or a regression of amyloidosis. In some embodiments, the method and/or system includes assessing a treatment program based on the identified changes, or lack thereof. In some embodiments, the subsequent profile of drusen is obtained following the initiation of a treatment program.

In some embodiments of the presently-disclosed subject matter, the profile of drusen is obtained by scanning the periphery of the retina. In some embodiments, the profile of drusen is obtained using a machine that allows for ultra-wide field imaging (UWFI) of a retina. In some embodiments, the profile of drusen is obtained using a wide angle ophthalmoscope.

In some embodiments of the presently-disclosed subject matter, the profile of drusen is a profile of hard drusen. In some embodiments, the amyloidosis is an amyloidosis of the CNS. In some embodiments, the amyloidosis is Alzheimer's disease.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 is a diagram of a system for predicting an amyloidosis disease status, in accordance with the presently-disclosed subject matter.

FIG. 2 includes typical ultra wide field images of AD patients. (A) The appearance of small hard drusen in the far periphery is strongly associated with AD. Detailed analysis of the far periphery in AD patients is often limited by artifacts introduced by inability to comply with this novel type of imaging, such as blinking artifacts, eyelashes and eyelids. (B): Characteristic small Drusen and pigmentary changes of the retinal periphery in AD.

FIG. 3 includes a bar graph generated by grading images for typical AMD categories such as hard drusen, soft drusen, geographic atrophy (GA) and coroidal neoascularisation (CNV). The only significant difference in peripheral retinal grading between controls and AD patients was detected in hard drusen. There was no CNV detected at the far periphery.

FIG. 4 includes a series of images. Image grading grid (A) for grading ultra-wide angle images. (B-H) show typical imaging of AD patients. (B) far peripheral hard drusen deposits. (C-D) images taken through cataract are still gradable. (E-F) typical lid and eye-lash artifacts.

FIG. 5 includes images showing extensive drusen deposition in the macula and periphery of flat-mounted human donor eye.

FIG. 6 includes a series of images, wherein the images in the first column show that normal macular appearance is accompanied by drusen on the periphery, the images in the second column snow choroidal neovascularisation (CNV) and atrophy in the macula with extensive drusen deposition and atrophy in the periphery, and the images in the third column show geographic atrophy (GA) in the macula with extensive drusen deposition and GA in the periphery.

FIG. 7 illustrates the prevalence of drusen found in various locations of the retina.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes methods and systems useful for predicting an amyloidosis, especially Alzheimer's disease status in a subject. The present inventors surprisingly discovered that drusen in the periphery of a subject's retina is/are useful for predicting an amyloidosis status in a subject. As will be known to those of ordinary skill in the art, drusen are extracellular deposits between the retinal pigment epithelium and the choroidal vasculature. Drusen imaging is fast, requires minimal co-operation on the part of the subject, is inexpensive in comparison to PET and MRI technology, and can be used for serial sampling. Recent technological advances in ultra-wide field imaging (UWFI) allow identification of peripheral retinal abnormalities, such as drusen in the periphery. Drusen are seen as yellowish spots external to the neural retina and readily analyzable. Furthermore, unlike deposits such as amyloid beta plaques, drusen can be seen without staining, and therefore can be detected with minimal invasiveness.

Methods and systems of the presently-disclosed subject matter include obtaining a profile of drusen in a periphery of a retina of a subject, and predicting an amyloidosis status based on the profile. In some embodiments the presently-disclosed subject matter includes, identifying a subject, the amyloidosis status of which is desired; and obtaining a profile of drusen in a periphery of a retina of the subject.

As used herein, the term “subject” includes both human and animal subjects. Thus, veterinary therapeutic uses are provided in accordance with the presently disclosed subject matter. As such, the presently disclosed subject matter provides for the treatment of mammals such as humans, as well as those animals of importance (e.g., due to being endangered, of economic importance, of social importance, etc.), including, but not limited to primates. Alternatively, the subject may be a mammal or bird used as an animal model, thus providing diagnostic uses, and methods of testing for compounds useful for treating amyloidosis. Such subjects include rodents, especially mice, rats and rabbits, larger mammals, such as dogs, pigs, cats and sheep, or even birds such as quail. When the subject is a mouse, it may be an APPS1 mouse.

As used herein, “profile of drusen” or “drusen profile” can refer to a qualitative assessment of drusen, such as a presence or absence of drusen, or a quantitative assessment of drusen, such as the number of drusen, area covered by drusen or a size of drusen. The term can also be used to refer to a pattern of drusen, including relative location of drusen, distribution of drusen, and the like. In some embodiments, the profile of drusen is a profile of hard drusen. As will be recognized by those of ordinary skill in the art, hard drusen can be differentiated from other drusen (i.e., soft or intermediate drusen) principally by size and by morphology. Hard drusen are less than 63 μm in diameter and have a sharper edge appearance. (1: Bird A C, Bressler N M, Bressler S B, Chisholm I H, Coscas G, Davis M D, de Jong P T, Klaver C C, Klein B E, Klein R, et al. (1995) An international classification and grading system for age-related maculopathy and age-related macular degeneration. The International ARM Epidemiological Study Group. Surv Ophthalmol 39, 367-374. 2: Ferris F L, Davis M D, Clemons T E, Lee L Y, Chew E Y, Lindblad A S, Milton R C, Bressler S B, Klein R (2005) A simplified severity scale for age-related macular degeneration: AREDS Report No. 18. Arch Ophthalmol 123, 1570-1574. The profile of hard drusen may include, for example, the number, size or distribution of hard drusen. It may also include comparing the number, size or distribution of hard drusen in comparison with the number of intermediate or soft drusen present in the periphery. Intermediate drusen are between 63 μm and 125 μm in diameter. Soft drusen are greater than 125 μm in diameter.

The term “obtain” or “obtaining”, when used with reference to a profile of drusen, refers to an action of collecting information regarding drusen in a retina of a subject. Such collection of information can include, for example, scanning for and/or capturing an image of drusen in the retina, which can be stored, if desired. For another example, drusen in the retina can be visualized and information collected by such visualization can be considered or processed, without a need to capture an image or otherwise store a record of such information. In some embodiments, obtaining a profile can refer to a second party receiving the profile from a first party who directly collects information regarding drusen in the retina of the subject. Because drusen in a retina of a subject cannot be seen by a naked human eye of an observer, it is necessary to make use of a technological aide, such as an appropriate machine that allows for ultra-wide field imaging (UWFI) of a retina. For example, a wide-angle ophthalmoscope could be used in accordance with the presently-disclosed subject matter. In some embodiments, the wide angle ophthalmoscope is up to 200°, 220° or even 240°. An example of an ophthalmoscope that could be used is the OPTOMAP P200C AF ultra wide-field scanning laser ophthalmoscope. Alternatively, a standard ophthalmoscope having a field of up to 50° or 70° may be used, it being focussed on the periphery of the retina, rather than on the macula. Such ophthalmoscopes and the use thereof to image the periphery are described in Seddon J M, Reynolds R, Rosner B (2009) Peripheral retinal drusen and reticular pigment: association with CFHY402H and CFHrs1410996 genotypes in family and twin studies. Investigative ophthalmology & visual science 50, 586-591

As noted herein, drusen in the periphery of the retina of a subject are of interest. As used herein, the term “periphery” refers to the area of the retina peripheral to the vascular arcade. The term vascular arcade is well known in the art and in most humans is a field of around 30° around the fovea. The term “periphery” may refer to the entire periphery or to a section or part thereof, especially to one half or one or more quadrants thereof. The method may include comparing one quadrant with another. When a section of the periphery is imaged, it is preferably the nasal half or supero half or the superonasal quadrant that is imaged.

As noted herein above, the presently-disclosed subject matter can be used to predict an amyloidosis status. An amyloidosis status can refer to, for example, a diagnosis of amyloidosis a diagnosis of no amyloidosis, a prognosis of amyloidosis, a risk associated with amyloidosis, e.g., a risk of contracting amyloidosis, a change in amyloidosis, a progression of amyloidosis, a regression of amyloidosis, and an absence of a change in amyloidosis. Amyloidosis status can be determined using one profile of drusen of a subject, or multiple profiles of drusen of a subject, e.g., serial screening.

Amyloidosis is a disease characterised by the presence of amyloids. As is known in the art, amyloids are extracellular deposits, seen in a number of diseases, such as Alzheimer's disease, Parkinson's disease, Huntington's disease, Transmissible spongiform encephalopathy, Diabetes mellitus type 2, Medullary carcinoma of the thyroid, Cardiac arrhythmias, Isolated atrial amyloidosis, Atherosclerosis, Rheumatoid arthritis, Aortic medial amyloid, Prolactinomas, Familial amyloid polyneuropathy, Hereditary non-neuropathic systemic amyloidosis, Dialysis related amyloidosis, Finnish amyloidosis, Lattice corneal dystrophy, Cerebral amyloid angiopathy, Cerebral amyloid angiopathy (Icelandic type), systemic AL amyloidosis, and Sporadic Inclusion Body Myositis. In one embodiment, the amyloidosis is an amyloidosis of the CNS, especially a neurodegenerative disease, such as Alzheimer's disease, Parkinson's disease, Huntington's disease and Transmissible spongiform encephalopathy. In particular the amyloidosis may be Alzheimer's disease.

As will be recognized by those skilled in the art upon study of this application, in some cases it can be useful to obtain multiple drusen profiles of a subject at different time points, selected based on the desired results. For example, such serial screening or monitoring can provide insight into change in amyloidosis status of a subject. Such serial screening can provide insight into change in amyloidosis status of a subject in the presence of a treatment program, and such change (or lack of change) in amyloidosis status can provide information useful for making decisions regarding the efficacy of the treatment program and/or changes to be made to the treatment program. Such treatment programs can include various components, including, but not limited to, administration of pharmaceutical or biologic formulations for the treatment of amyloidosis.

In some embodiments, the presently-disclosed subject matter includes obtaining a profile of drusen in the periphery of a retina of a subject, obtaining a subsequent profile of drusen in the periphery of the retina of the subject, and predicting an amyloidosis status based on one or more of the drusen profiles and/or change in the drusen profiles.

As will be recognized by those skilled in the art upon study of this application, it can be useful to predict an amyloidosis status using the profile of drusen, with reference to one or more controls. A control refers to a known amyloidosis status. In some embodiments, the control can be a profile of drusen associated with a known amyloidosis status, such as a profile of an individual or a composite profile of individuals of a known amyloidosis status. In some cases, it can be desirable to make use of multiple controls, each associated with a distinct known amyloidosis status. For example, in some embodiments it can be useful to compare the profile of the subject with a first control profile associated with a first known amyloidosis status, and a second control profile associated with a second known amyloidosis status. The known amyloidosis status control or controls selected can be any useful control or controls under the particular circumstances, including, but not limited to, the following examples: known diagnosis of amyloidosis, diagnosis of no amyloidosis, prognosis of amyloidosis, risk associated with amyloidosis, stage of amyloidosis, change in amyloidosis, progression of amyloidosis, regression of amyloidosis, and absence of a change in amyloidosis.

Predicting an amyloidosis status according to the presently-disclosed subject matter can refer to making an assessment regarding risk, diagnosis, and/or prognosis. The terms diagnosing and diagnosis refer to methods by which the skilled artisan can estimate and even determine whether or not a subject is suffering from amyloidosis. The terms prognosing and prognosis refer to predicting a clinical outcome (with or without medical treatment), selecting an appropriate treatment (or whether treatment would be effective), monitoring a current treatment and potentially changing the treatment, based on a profile of drusen and/or a change in drusen profile.

The term “predicting” does not refer to the ability to make a prediction with 100% accuracy, or even that a particular result is predictably more or less likely to occur based on the profile. Instead, the skilled artisan will understand that the term “predicting” refers to an increased probability that a certain course or outcome will occur; that is, that a course or outcome is more likely to occur in a subject exhibiting a particular profile, when compared to those individuals not exhibiting the profile. For example, in individuals not exhibiting the condition (e.g., profile including a substantial absence of drusen), the chance of a given outcome (e.g., suffering from amyloidosis) may be very low (e.g., <1%), or even absent. In contrast, in individuals exhibiting the condition (e.g., including a presence of drusen or including an amount, size, distribution, or pattern of drusen distinct from a normal control), the chance of a given outcome (e.g., suffering from amyloidosis) may be high. In certain embodiments, a prediction is about a 5% chance of a given expected outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, or about a 95% chance.

The skilled artisan will understand that associating a drusen profile with a predisposition to an outcome can include a statistical analysis. For example, a particular profile of drusen that differs from a control in terms of presence, amount, size, distribution, ratio of type of hard drusen to soft drusen and/or pattern of drusen in some embodiments can signal that a subject is more likely to suffer from amyloidosis than subjects with a profile that is a level less than or equal to the control level, as determined by a level of statistical significance. For example, a particular profile of drusen that includes a presence of drusen in some embodiments can signal that a subject is more likely to suffer from amyloidosis than subjects with a profile includes an absence of drusen, as determined by a level of statistical significance. Statistical significance is often determined by comparing two or more populations, and determining a confidence interval and/or a p value. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001.

In some embodiments of the presently-disclosed subject matter a system is provided for predicting an amyloidosis status, including a machine for obtaining a profile of drusen in a periphery of a retina of a subject; and a control or plurality of controls of amyloidosis status. As described hereinabove, the machine can be a machine that allows for ultra-wide field imaging (UWFI) of a retina. For example, a wide-angle ophthalmoscope could be used in accordance with the presently-disclosed subject matter. In some embodiments, the wide angle ophthalmoscope is up to 200°, 220° or 240°. An example of an ophthalmoscope that could be used is the OPTOMAP P200C AF ultra wide-field scanning laser ophthalmoscope. A control or plurality of controls is provided together with the machine, to which an obtained profile of a subject can be compared for use in predicting an amyloidosis status. Such control or plurality of controls can comprise, for example, a record of images or other information about profiles associated with known amyloidosis status. In some embodiments, the control or plurality of controls can be stored on data storage means and/or can be associated with amyloidosis status program modules.

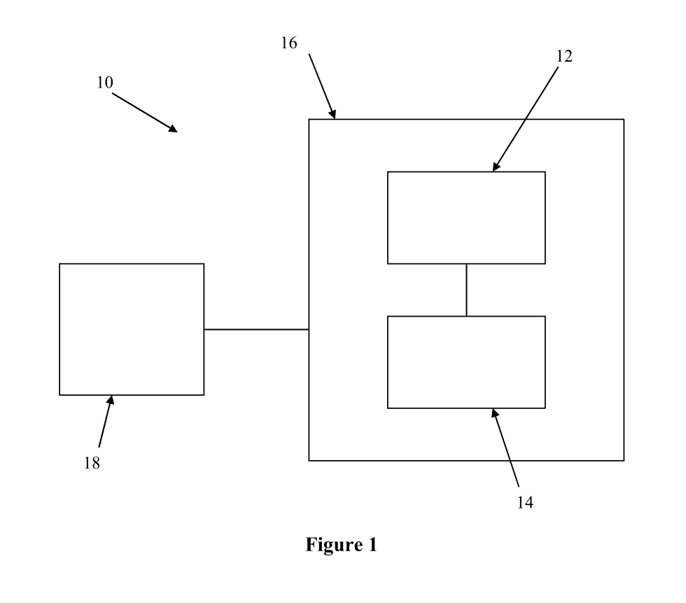

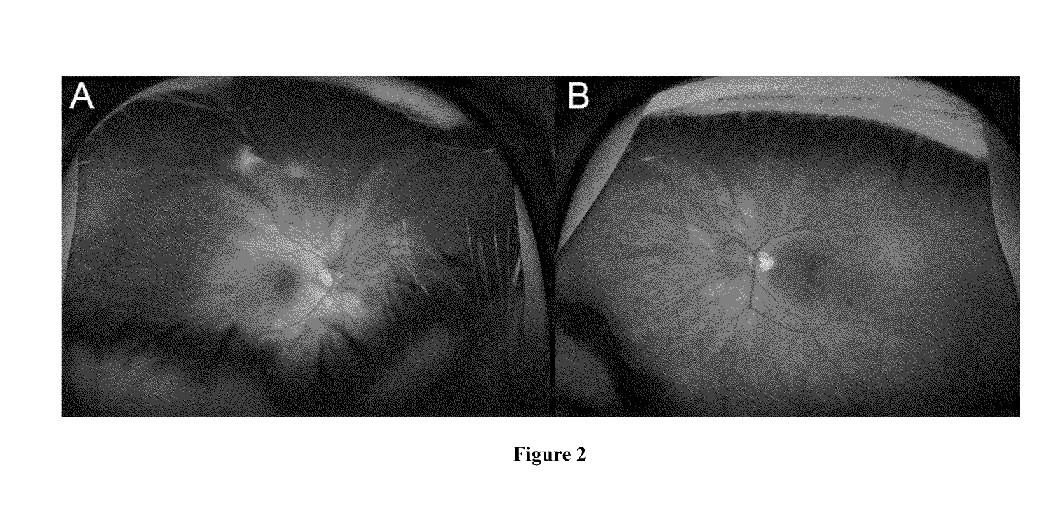

With reference to FIG. 1, in some embodiments of the presently-disclosed subject matter a system 10 is provided for predicting an amyloidosis status, including data storage means 12; amyloidosis status program modules stored on the data storage means 12; a processor means 14 executing the amyloidosis status program modules: obtaining a profile of drusen in a periphery of a retina of a subject; and predicting an amyloidosis status based on the profile. A general-purpose computer or other appropriate machine 16 can comprise the data storage means 12 and processor means 14. In some embodiments, the system 10 can further include a machine 18 for obtaining a profile of drusen in a periphery of a retina of a subject, such as a machine that allows for ultra-wide field imaging (UWFI) of a retina.

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by one of ordinary skill in the art, definitions are set forth herein to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth. Conversely, when a term referencing a plurality is used, it is inclusive of a singularity. Thus, for example, reference to “drusen” includes “a druse”, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter is further illustrated by the following specific but non-limiting examples. The following examples may include compilations of data that are representative of data gathered at various times during the course of development and experimentation related to the present invention.

### EXAMPLES

### Example 1

Ultra-Wide Field Retinal Imaging of Alzheimer's Disease.

Alzheimer's disease (AD) and age-related changes of the retina are both characterized by extracellular deposit formation: plaques in the brain and drusen in the retina. In the study described in this example, it was examined whether there is a direct association between deposit formation under the retina and AD by imaging 55 AD subjects and 48 controls using an ultra-wide field (up to 200°) laser scanning ophthalmoscope. A significant association between AD and peripheral hard drusen was found. These results support the efficacy of monitoring the development and progression of pathological changes in the retinal periphery for monitoring and diagnosing AD.

Introduction. Amyloid plaques, and other characteristic pathological signs of Alzheimer's disease (AD), are known to develop many years before the onset of cognitive symptoms[1, 2]. However, tests for AD before dementia develops are of unproven accuracy, too expensive or too invasive for routine clinical use[3]. Drusen in the outer retina share many similarities to extracellular deposits seen in AD. Firstly they appear early in association with ageing[4]. Secondly, they contain amyloid beta protein and a variety of identical macromolecules[5 ] and finally they contain micro elements, such as zinc which might have special relevance for both diseases[6, 7]. Unlike the extracellular deposits seen in AD, drusen are readily detected through non-invasive retinal imaging. Analysis of the macula of AD sufferers for size and distribution of Drusen, has suggested a possible association with AD severity[8]. However, Drusen deposition is more widespread in the retinal periphery than in the macula[4]. The study described in this example examined whether peripheral retinal Drusen would be more common in AD subjects than non-demented controls, and contemplates use of peripheral Drusen as an accessible surrogate marker for the amyloid development in the brain in Alzheimer's and elsewhere in other forms of amyloidosis.

Materials and Methods. AD subjects had their diagnosis confirmed by trained clinicians using NINCDS-ADRDA criteria[9]. Controls were drawn from a database of carers from a single cognitive disorders clinic where the study took place. Data were collected on medical history, genotype and standard socio-demographic factors which may confound the relationship between AD and the presence of Drusen. The study proceeded after local ethics approval was secured.

Following consent, participants were photographed using the OPTOMAP P200C AF ultra wide-field (up to 200°) scanning laser ophthalmoscope that was operated by an Imaging Team provided by OPTOS Plc and supervised by the Reading Centre of Moorfields Eye Hospital (MEHRC). Blood for genotyping and a clinical history were also taken. Image analysis was completed by readers masked to the participants' case-control status. Images were graded randomly in the macula and the periphery following the automatic fitting of a standard macular grid defined by the distance between the centres of the optic nerve head and the fovea[10]. Only abnormalities resembling age related changes like hard and soft Drusen, retinal pigment epithelial changes, pigment epithelial detachment, geographic atrophy and chorioretinal neovascularisation were graded[10]. Pupils were dilated with 1% tropicamide (Tropicamide) drops (Bausch & Lomb) if required.

In a previous study[8] 11.5% of subjects had Drusen in their macula which was taken as an estimate for peripheral Drusen numbers in controls. To demonstrate a 3-fold increase in peripheral Drusen prevalence in AD, with α of 0.05 and a power of 80%, then 50 subjects were needed in each arm. Analysis was undertaken in SPSS 14.0, (Chicago: SPSS Inc). The crude association between AD status and the presence of Drusen was determined using the chi-squared test. Nominal regression was used to adjust the crude associations by potential confounders selected if their association with both AD status and retinal pathology generated a p-value of <0.1.]

Results. One hundred and seven participants (59 AD subjects and 48 controls) had retinal examination conducted over a 3 day period. Four subjects had their data excluded from analysis (3 with Lewy Body Dementia and one due to non compliance) giving a final analysis set of 55 subjects and 48 controls. AD subjects were more likely to need their pupils dilated to take photographs (χ²=33.2, df=1, p<0.001) though this did not affect the grading of the images. AD subjects were significantly more likely to have hard drusen phenotype in the periphery [14/55 (25.4%) compared to 2/48 (4.2%) of controls; χ²=9.9, df=4, p=0.04] (Table 1). There was no other significant association of retinal changes with AD (Table 1). The following factors were associated with AD: ApoE4 RS429358 status (χ²=4.9, df=2, p=0.09), a history of TIA (χ²=3.1, df=1, p=0.08) and being an ex-smoker (χ²=5.6, df=2, p=0.06). AD subjects were also older [AD=79.2 (SD=8.4); Controls=70.7 (SD=10.4); p<0.0001]. Peripheral hard Drusen were associated with history of heart failure (χ²=8.1, df=4,p=0.09), stroke (χ²=10.1, df=4,p=0.04) and TIA (χ²=8.1, df=4, p=0.09). Given the strength of the association between age and AD and the potential confounding effect of TIA's, the model was reanalyzed taking account of these variables. This adjusted analysis demonstrated a very strong association between the presence of peripheral hard Drusen and AD (χ²=32.3, df=12, p=0.001).

TABLE 1
Analysis of peripheral retinal phenotype by AD/Control status.

| | | AD | Controls | | | |
|---|---|---|---|---|---|---|
| | | N (%) | N (%) | χ² | df | p-valu [symbol] |
| Peripheral | Normal | 20 (35.7%) | 21 (43.8%) | | | |
| Phenotype | Hard Drusen | 14 (25%) | 2 (4.2%) | | | |
| | Soft Drusen | 18 (32.1%) | 22 (45.8%) | | | |
| | Atrophy | 3 (5.3%) | 3 (6.2%) | | | |
| | Ungradable | 1 (1.7%) | 0 (0%) | 9.9 | 4 | 0.04 |

[symbol]

indicates data missing or illegible when filed

Discussion. The association of deposit formation in the peripheral retina with AD was investigated using an ultra-wide field laser scanning ophthalmoscope. A highly significant association between peripheral hard Drusen deposition and AD was found, supporting the utility of method for detecting, diagnosing, and/or monitoring AD by identifying peripheral hard Drusen. Identification of peripheral hard Drusen is also contemplated as an outcome measure for disease modifying agents in clinical trials for AD. The tantalizing prospect that drugs that modify the course of AD through an impact on Aβ pathology can also reduce the risk of developing blindness due to AMD should also be considered.

### Example 2

Peripheral Retinal Drusen as a Surrogate Marker for Alzheimer's Dementia: A Pilot Ultra-Wide Angle Imaging Study.

Purpose: The development of Alzheimer's dementia (AD) and age-related macular degeneration (AMD) share similar histopathology, vascular risk factors and genetic predisposition and both are characterized by extracellular deposit formation. In the study described in the present example, the prevalence and spatial distribution of macular and peripheral retinal pathologies in patients with AD were examined.

Methods: Colour and autofluorescent (AF) images were taken by the Optos P200C AF ultrawide angle laser scanning ophthalmoscope (200°) to determine phenotypic variations in 56 AD patients and 46 controls. Images were graded for the prevalence of drusen, pigmentary changes, atrophy or choroidal neovascularisation (CNV) in the macula as well as retinal periphery. The periphery was divided into two zones (zone 4 and 5) to extend the standard AMD grid and pathological distribution was recorded in four sectors within these zones. All subjects had blood taken for genotyping. Comparisons were made using the chi-squared test after adjustment for potential confounders.

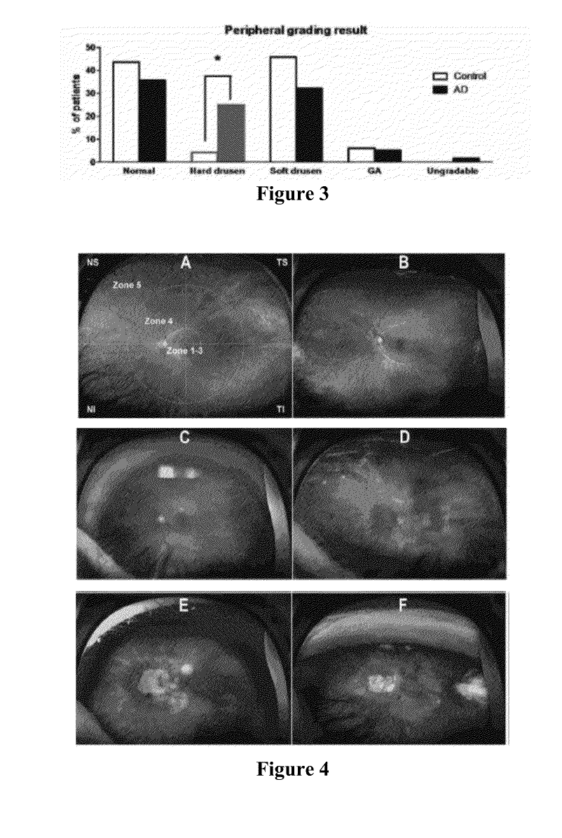

Results: With reference to FIGS. 3 and 4, there was a positive genetic association between AD and ApoE4 RS429358 (p=0.09). Only one control, but 4 patients were diagnosed to have AMD based on macular pathologies. In the periphery hard drusen were present in 14/55 (25.4%) of AD patients and 2/48 (4.2%) of controls (Chi²=9.9, df=4, p=0.04]. After adjustment for age and history of transient ischemic attack, this association remained strongly significant (p<0.001).

Discussion: Ultra-wide angle imaging revealed a potential association between AD and AMD and a highly significant association between AD and peripheral hard drusen formation. These findings suggest that monitoring for the development and progression of pathological changes in the macula and most importantly in the periphery might become a valuable tool in detecting and monitoring the progression of AD. Further work is required to develop the understanding of this association which may lead to peripheral drusen acting as a surrogate marker for plaque development in the central nervous system.

### Example 3

AMD-like Pathologies at the Peripheral Retina in the Reykjavik Eye Study: An Ultra Wide-angle (200°) Colour and Auto Fluorescent Digital Image Grading Study.

Purpose: The understanding of the relevance of peripheral retinal pathologies in general, and in Age Related Macular Degeneration (AMD) in particular, is limited by the lack of detailed peripheral imaging studies. Imaging retinal periphery in clinical setting is now available; thus the purpose of this study was to develop imaging and grading protocols suited to wide-angle imaging in an aged population.

Methods: Ultra wide field (200°) color and autofluorescence (AF) images were taken of 573 participants using Optos P200C AF ultra-wide angle laser scanning ophthalmoscope as part of the 12 year follow-up of the Reykjavik Eye Study, a random population sample. The images were graded at Moorfields Eye Hospital Reading Centre. Peripheral changes were graded using a standardised grid developed for this imaging modality. Presence or absence of hard and soft drusen, peripheral retinal pigment epithelial changes, atrophy or neovascularisation was graded on the color images and then the presence or absence of hypo-and hyperfluorescence using autofluorescent images. All peripheral grading results were tabulated and then the presence of the same changes was examined for the macula and the peripheral grading.

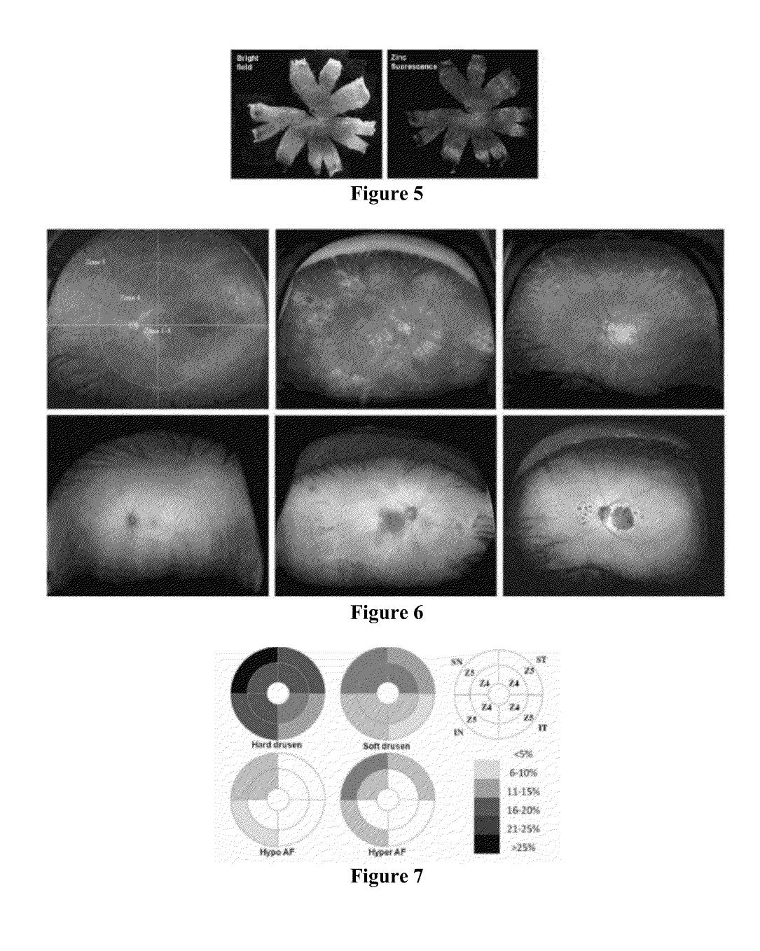

Results: Reference is made to FIGS. 5-7. Participants were 62 years and older and 18.9% of the eyes examined were free of pathologies both in the macula and the periphery. There were eyes with pathology only in the macula (13.6%) or only in the periphery (10.1%) while pathologies at both locations were the most frequent (57.4%), with the majority of pathologies being in the far periphery (zone 5). More Drusen were observed in the superior 2 quadrants, compared to the inferior ones. There was no AMD-like CNV or PED in the periphery except in those cases where these pathologies clearly originated from the macula. Seven patients had AMD-like geographic atrophy at the periphery without end-stage disease in the macula. No patient with end-stage disease in the macula had normal periphery. In a few cases (up to 3% of total) there were pathologies on AF but not on colour images.

Discussion: Phenotyping retinal periphery confirmed wide ranging pathological changes even in those without central sight threatening disease. The association of peripheral and central pathologies are yet to be determined in follow up studies. It appears that imaging AF in the periphery may aid diagnoses in addition to its value of detecting RPE abnormalities.

It will be understood that various details of the presently-disclosed subject matter can be changed without departing from the scope of the subject matter disclosed herein. Furthermore, the foregoing description is for the purpose of illustration only, and not for the purpose of limitation.

### Incorporation by Reference

All publications, patents, and patent applications mentioned in this specification, including those set forth in the following list, are herein incorporated by reference to the same extent as if each individual publication, patent, or patent application was specifically and individually indicated to be incorporated by reference.

### REFERENCES

[1] Scahill R I, Schott J M, Stevens J M, Rossor M N, Fox N C (2002) Mapping the evolution of regional atrophy in Alzheimer's disease: unbiased analysis of fluid-registered serial MRI. Proc Natl Acad Sci USA 99, 4703-4707.

[2] Hampel H, Burger K, Teipel S J, Bokde A L, Zetterberg H, Blennow K (2008) Core candidate neurochemical and imaging biomarkers of Alzheimer's disease. Alzheimers Dement 4, 38-48.

[3] Mason S E, McShane R, Ritchie C W (2010) Diagnostic tests for Alzheimer's disease: rationale, methodology, and challenges. Int J Alzheimers Dis 2010.

[4] Lengyel I, Tufail A, Hosaini H A, Luthert P, Bird A C, Jeffery G (2004) Association of drusen deposition with choroidal intercapillary pillars in the aging human eye. Invest Ophthalmol Vis Sci 45, 2886-2892.

[5] Hageman G S, Luthert P J, Victor Chong N H, Johnson L V, Anderson D H, Mullins R F (2001) An integrated hypothesis that considers drusen as biomarkers of immune-mediated processes at the RPE-Bruch's membrane interface in aging and age-related macular degeneration. Prog Retin Eye Res 20, 705-732.

[6] Bush A I (2002) Metal complexing agents as therapies for Alzheimer's disease. Neurobiol Aging 23, 1031-1038.

[7] Lengyel I, Flinn J M, Peto T, Linkous D H, Cano K, Bird A C, Lanzirotti A, Frederickson C J, van Kuijk F J (2007) High concentration of zinc in sub-retinal pigment epithelial deposits. Exp Eye Res 84, 772-780.

[8] Klaver C C, Ott A, Hofman A, Assink J J, Breteler M M, de Jong P T (1999) Is age-related maculopathy associated with Alzheimer's Disease? The Rotterdam Study. Am J Epidemiol 150, 963-968.

[9] McKhann G, Drachman D, Folstein M, Katzman R, Price D, Stadlan E M (1984) Clinical diagnosis of Alzheimer's disease: report of the NINCDS-ADRDA Work Group under the auspices of Department of Health and Human Services Task Force on Alzheimer's Disease. Neurology 34, 939-944.

[10] Bird A C, Bressler N M, Bressler S B, Chisholm I H, Coscas G, Davis M D, de Jong P T, Klaver C C, Klein B E, Klein R, et al. (1995) An international classification and grading system for age-related maculopathy and age-related macular degeneration. The International ARM Epidemiological Study Group. Surv Ophthalmol 39, 367-374.

[11] Jack C R, Jr., Knopman D S, Jagust W J, Shaw L M, Aisen P S, Weiner M W, et al. Hypothetical model of dynamic biomarkers of the Alzheimer's pathological cascade. Lancet Neurol. 2010 January; 9(1):119-28.

[12] Vingerling J R, Dielemans I, Hofman A, Grobbee D E, Hijmering M, Kramer C F, et al. The prevalence of age-related maculopathy in the Rotterdam Study. Ophthalmology. 1995 February; 102(2):205-10.

[13] Lengyel I, Tufail A, Hosaini H A, Luthert P, Bird A C, Jeffery G. Association of drusen deposition with choroidal intercapillary pillars in the aging human eye. Invest Ophthalmol Vis Sci. 2004 September; 45(9):2886-92.

[14] Ritchie C W, Peto T, Barzegar-Befroei N, Csutak A, Ndhlovu P, Wilson D, et al. Peripheral Retinal Drusen as a Potential Surrogate Marker for Alzheimer's Dementia: A Pilot Study Using Ultra-Wide Angle Imaging. Invest Ophthalmol Vis Sci. 2011 Apr. 22, 2011; 52(6):6683.

[15] Johnson L V, Leitner W P, Rivest A J, Staples M K, Radeke M J, Anderson D H. The Alzheimer's A beta-peptide is deposited at sites of complement activation in pathologic deposits associated with aging and age-related macular degeneration. Proc Natl Acad Sci U S A. 2002 Sep. 3; 99(18):11830-5.

[16] Hageman G S, Luthert P J, Victor Chong N H, Johnson L V, Anderson D H, Mullins R F. An integrated hypothesis that considers drusen as biomarkers of immune-mediated processes at the RPE-Bruch's membrane interface in aging and age-related macular degeneration. Prog Retin Eye Res. 2001 November; 20(6):705-32.

[17] Lengyel I, Flinn J M, Peto T, Linkous D H, Cano K, Bird A C, et al. High concentration of zinc in sub-retinal pigment epithelial deposits. Exp Eye Res. 2007 April; 84(4):772-80.

[18] Nan R, Farabella I, Schumacher F F, Miller A, Gor J, Martin A C, et al. Zinc binding to the tyr402 and his402 allotypes of complement factor h: possible implications for age-related macular degeneration. J Mol Biol. 2011 May 13; 408(4):714-35.

What is claimed is:

1. A method of predicting an amyloidosis status, comprising:
(a) obtaining a profile of drusen in a periphery of a retina of a subject; and using the profile to predict an amyloidosis status;
(b) identifying a subject, the amyloidosis status of which is desired; and obtaining a profile of drusen in a periphery of a retina of the subject;
(c) analyzing a profile of drusen in a periphery of a retina of a subject to predict an amyloidosis status of the subject; or (d) comparing a profile of drusen in a periphery of a retina of a subject to a control;
and predicting an amyloidosis status of the subject.

2. The method of claim 1, comprising
identifying a subject, the amyloidosis status of which is desired;
obtaining a profile of drusen in a periphery of a retina of the subject; and
using the profile to predict an amyloidosis status.

3. The method of claim 1, comprising
analyzing a profile of drusen in a periphery of a retina of a subject to predict an amyloidosis status of the subject; and
using a system for predicting an amyloidosis status, comprising: data storage means;
amyloidosis status program modules stored on the data storage means; a processor means executing the amyloidosis status program modules: obtaining a profile of drusen in a periphery of a retina of a subject; and predicting an amyloidosis status based on the profile.

4. The method of claim 1, comprising
comparing a profile of drusen in a periphery of a retina of a subject to a control;
predicting an amyloidosis status of the subject; and
using a system for predicting an amyloidosis status, comprising: data storage means;
amyloidosis status program modules stored on the data storage means; a processor means executing the amyloidosis status program modules: obtaining a profile of drusen in a periphery of a retina of a subject; and predicting an amyloidosis status based on the profile.

5. The method of claim 1, wherein predicting an amyloidosis status comprises making a diagnosis.

6. The method of claim 1, wherein predicting an amyloidosis status comprises making prognosis.

7. The method of claim 1, wherein predicting an amyloidosis status comprises making an assessment of risk.

8. The method of claim 1, wherein the profile comprises a quantitative assessment of drusen in the periphery of the retina of the subject.

9. The method of claim 1, wherein the profile comprises a qualitative assessment of drusen in the periphery of the retina of the subject.

10. The method of claim 1, wherein the profile comprises a presence or absence of drusen in the periphery of the retina of the subject.

11. The method of claim 1, wherein the profile comprises an amount of drusen in the periphery of the retina.

12. The method of claim 1, wherein the profile comprises a size of drusen in the periphery of the retina.

13. The method of claim 1, wherein the profile comprises a pattern of drusen in the periphery of the retina.

14. The method of claim 1, wherein the profile comprises a location of drusen in the periphery of the retina.

15. The method of claim 1, wherein the profile comprises a distribution of drusen in the periphery of the retina.

16. The method of claim 1, and further comprising comparing the profile to a control.

17. The method of claim 16, wherein the control comprises a profile of drusen associated with a known amyloidosis status.

18. The method of claim 17, wherein the known amyloidosis status is a diagnosis of amyloidosis.

19. The method of claim 17, wherein the known amyloidosis status is a diagnosis of no amyloidosis.

20. The method of claim 17, wherein the known amyloidosis status is a known stage of amyloidosis.

21. The method of claim 1, and further comprising obtaining a subsequent profile of drusen in the periphery of a retina of a subject.

22. The method of claim 21, and further comprising predicting an amyloidosis status based on the drusen profiles and/or change in the drusen profiles.

23. The method of claim 21, and further comprising comparing the profile and the subsequent profile.

24. The method of claim 21, and further comprising identifying changes, if any, between obtained profiles of drusen.

25. The method of claim 24, and further comprising predicting an amyloidosis status based on the identified changes, or lack thereof.

26. The method of claim 25, wherein the amyloidosis status is no substantial change, a progression of amyloidosis, or a regression of amyloidosis.

27. The method of claim 24, further comprising assessing a treatment program based on the identified changes, or lack thereof

28. The method of claim 21, wherein the subsequent profile of drusen is obtained following the initiation of a treatment program.

29. The method of claim 1, wherein the profile of drusen is obtained by scanning the periphery of the retina.

30. The method of claim 1, wherein the profile of drusen is obtained using a machine that allows for ultra-wide field imaging (UWFI) of a retina.

31. The method of claim 1, wherein the profile of drusen is obtained using a wide angle ophthalmoscope.

32. The method of claim 1, wherein the profile of drusen is a profile of hard drusen.

33. The method of claim 1, wherein the amyloidosis is an amyloidosis of the CNS.

34. The method of claim 1, wherein the amyloidosis is Alzheimer's disease.

35. A system for predicting an amyloidosis status, comprising:
(a) a machine for obtaining a profile of drusen in a periphery of a retina of a subject; and a control of amyloidosis status; or
(b) data storage means; amyloidosis status program modules stored on the data storage means; a processor means executing the amyloidosis status program modules, including obtaining a profile of drusen in a periphery of a retina of a subject; and predicting an amyloidosis status based on the profile.

36. The system of claim 8, comprising a machine for obtaining a profile of drusen in a periphery of a retina of a subject; data storage means; amyloidosis status program modules stored on the data storage means; a processor means executing the amyloidosis status program modules, including obtaining a profile of drusen in a periphery of a retina of a subject; and predicting an amyloidosis status based on the profile.
